(19) United States
(12) Patent Application Publication
(10) Pub. No.: US 2005/0033112 A1
(43) Pub. Date: Feb. 10, 2005

(54) Sexual aid device

(76) Inventors: Jeffrey Francis Bruton, Bridport, VT (US); Marco Ferrero, New York, NY (US)

Correspondence Address: James J. Merrick;Cantor Colburn LLP, 55 Griffin Road South, Bloomfield, CT, 06002, US

(21) Appl. No.: 10/637,764

(22) Filed: Aug. 8, 2003

(51) Int. Cl. A61F 5/00; A61H 1/00

(52) U.S. Cl. 600/38; 601/70

(57) ABSTRACT

A sexual aid device includes a housing having a generally cylindrical base and central portion and an upper portion defining a closed upper end. The base includes an open lower end and at least the base and central portion are hollow. The housing is fabricated of a lubricious glass-based single piece of material. An electric motor is mounted in the hollow central portion of the housing and vibrator means including a block and an eccentric drive therefore is coupled to the motor. A battery means is disposed in the hollow base of the housing and is operably coupled with the motor. The motor and battery means are connected in series circuit and an end sealing means including a cap mounted on the open end of the base for releasably sealing the open end so that the hollow interior of the housing is waterproof when the cap is mounted on the base. The end sealing means provides an external switch means operably coupled with the battery means and the motor for selectively energizing the motor to drive the block in eccentric rotation to thereby cause the housing to vibrate.

### TECHNICAL FIELD

The present invention relates to a new and improved sex aid and, more particularly, to the field of vibrators, including prosthetic devices and dildos.

### BACKGROUND

Phalluses have been used as penis substitutes from time immemorial. In cultures where hymenal blood was considered evil or dangerous, a husband would avoid his young bride until she attended a ceremony during which her hymen was pierced by a substitute for the husband, often a phallus made of stone, metal, ivory or even wood. In other cultures the deflowering of a young bride by the phallus of a fertility god was part of ceremonies aimed at assuring the procreative success of a married couple. Similar ceremonies were also participated in by long-married wives who were childless. See Panati's Extraordinary Endings of Practically Everything and Everybody, Charles Panati (Harper & Row, N.Y., 1989).

In addition to serving in sacred fertility and marriage ceremonies, phalluses have been used for the simple purpose of pleasure, by couples and by people who are otherwise alone. In addition, with the onslaught of venereal diseases such as herpes and AIDS, phalluses substitute for the male reproductive organ as part of safe sex practices. Phalluses are also used by couples when the male partner is impotent. And, sex aids and paraphernalia, such as phalluses, have been used by sex counselors as one of the tools for counseling their patients.

Even from earliest times phalluses have been made as simple as an ordinary smooth-edged cylinder, as close replicas of human penises, as reproductions of exaggerated erect penises, or as ornately decorated symbols of the male reproductive organ.

In more recent times, with the development of modern plastics and other moldable materials, phalluses may be mass produced in a wide variety of forms pleasing in shape and design for use as decorative sculptures, or as a device used in sexual activities.

Mass produced phalluses are typically formed in a molding process. First a hollow phallus body is formed. Then often a vibrating device connected to a wire is inserted into the hollow area of the phallus. Finally, the hollow area is filled in by pouring a melt of a resilient material into the hollow area and letting it cool and harden. There is then a trimming step, where excess material from the pouring and hardening stage is cut off from the phallus.

There are problems associated with this manufacturing technique, however. First, the motor within the vibrating device is sometimes flooded and ruined during the pouring stage. In addition, once the poured material has solidified, the vibrating device within the phallus is not always located in the proper, centered, position, because the vibrating device may have moved due to force from the pouring or from any jostling or movement of the entire phallus during pouring and cooling. In addition, the wire running from the vibrating device to the power supply is sometimes burned and damaged during the pouring stage due to the heat (typically 400° F.) of the melt. The wire can also be cut or damaged during the trimming stage.

When any of the above problems occur, the entire phallus itself is typically discarded, since it is not possible to align or make repairs to the vibrating device or wire once the melt has solidified.

Vibrators of the prior art are powered either by wall socket voltage or batteries. The former results in a device which would reach undesirable temperatures after prolonged use, not advisable for use near water, and mobility thereof limited to the length of the cord extending to the wall socket. The latter results in a device which is short-lived, unreliable, and subject to damage if used in water.

Vibrators of the prior art have not been sufficiently water-resistant. This frequently results in an apparatus which is used in an area that perspires, and can be contaminated with discharge and other bodily fluids but can not be submerged in soap and water for thorough cleaning. In extreme cases, the vibrator could not be used near water, such as a bathtub, shower, or hot tub, as may be present in some suggestive circumstances prescribed by the marital therapist.

Furthermore, polymeric vibrators of the prior art are not capable of sterilization, as they can not be cleaned at such high temperature with out melting. In addition, polymeric vibrators can not even be sanitized to an acceptable degree for use between different partners or made to be odorless, as they are somewhat porous. Moreover, polymeric vibrators of the prior art do not satisfactorily hold hot or cold temperatures, and/or lubricants applied thereto for increasing stimulation during sex acts.

Therefore, it can be appreciated that there exists a continuing need for a new and improved sex aid which can be used for increasing stimulation during sex acts, that is waterproof, and capable of being sterilized for use with different partners. In this regard, the present invention substantially fulfills this need.

### SUMMARY

All of the problems noted above are solved by the invention described in this application. There is no pouring of a melt into the phallus body hollow, so all problems associated with the position of the vibrating device, and the destruction of the motor and wire, due to the pouring or the heat of the melt are eliminated. Furthermore, in a preferable embodiment of the invention the vibrating device is held in place by the physical dimensions within the hollow and a sleeve fitting around a motor within a portion of the hollow, so the device is properly positioned and can withstand jostling during manufacture and intended use. In addition, the present invention does not require a methodology of the trimming step, so there is less chance that the wire will be damaged during manufacture. Furthermore, the present invention allows removal of the motor for inspection and/or replacement, as well as for sterilization of the glass structure defining the hollow.

The device of the present invention is a single piece body (except for the removable cap), preferably phallus shaped, fabricated of a generally lubricous glass-based material containing an appreciable amount of an oxide of boron to render it lubricious and resistant to heat, chemicals and electricity defining the hollow therein. A motor and spinner are positioned within the hollow and preferably held in such position by virtue of the dimensions of a casing impression portion of the hollow, and a sleeve which is inserted around at least the motor to wedge against the hollow.

The motor and spinner are preferably enclosed in a casing, which is then inserted into the casing impression portion of the hollow region within the body. The casing impression portion is preferably an impression within the hollow area of the body which generally corresponds to the dimensions of the casing. The sleeve is preferably made of a resilient material and conforming to the dimensions of a portion of the hollow in which it is then inserted into the hollow of the body to thereby preferably close the hollow and prevent the casing from coming out of the hollow.

Optionally, another sleeve may be disposed around a battery housing that is operably coupled with the motor opposite the first sleeve. The second sleeve then can fill a space between the battery housing and the inner surface of the body defining the hollow. The cross sectional diameter of a resilient cap enclosing the motor and battery housing is preferably larger than that of the hollow. One or both of the sleeves may be formed of a resilient material or a material having a durometer hardness of about 50 to about 100. As a result when the cap enclosing the motor and battery casing assembly is forced into the hollow, a watertight fit is obtained between the cap and the body. Alternatively, the cap may be configured to enclose the motor and battery housing assembly in the hollow of the body by compressive fit around an exterior perimeter defining the body.

It is therefore an object of the present invention to provide a new and improved sex aid which has all the advantages of the prior art sex aids of known designs and configurations and none of the disadvantages.

It is another object of the present invention to provide a new and improved sex aid which may be easily and efficiently manufactured and marketed.

It is a further object of the present invention to provide a new and improved sex aid which is of a durable, odor free, and reliable construction.

It is yet a further object of the present invention to provide a new and improved sex aid which may be readily sterilized for improved sanitation.

Still another object of this invention is to provide a strong, rugged and durable cordless electric vibrator for use on the human body of the character described which shall be watertight, relatively inexpensive to manufacture, easy to assemble, and which shall yet be practical and efficient to a high degree in use.

Yet another object of the invention is to provide a glass housing for use with prior art vibrators such that a prior art vibrator may be safely used by different partners, while the housing and removable cap allow removal of the vibrator for cleaning the glass housing for such use.

Lastly another object of the invention is to provide a cordless vibrator which may be selectively operated at low or high speed, as desired by the user, by simple manual, rotation of the end cap.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there is illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a front perspective view of an exemplary embodiment of a sexual aid device;

FIG. 2 is an exploded view of the exemplary embodiment of FIG. 1;

FIG. 3 is a cross-sectional view of another exemplary embodiment of a sexual aid device illustrating a threadable cover; and

FIG. 4 is a cross-sectional of another alternative exemplary embodiment of a sexual aid device having two batteries.

### DETAILED DESCRIPTION

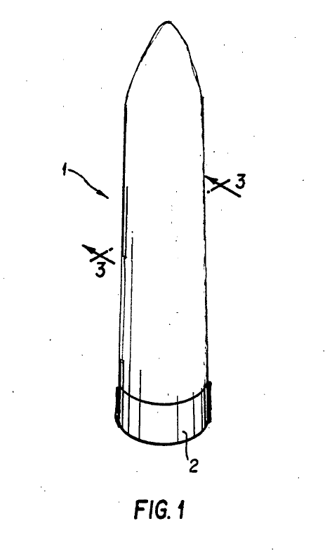

In exemplary embodiments as shown in FIGS. 1, 2, and 3, a sleeve or body 1 of this invention is arranged to provide a hollow and conical configuration for enclosing a motor 4, an eccentric block 41, and for holding a battery 5 therein. Body 1 is configured as an elongated housing having a generally cylindrical base and central portion and may include a generally conical upper portion or head. However, it is contemplated that upper portion or head may assume a number of different shapes and is not limited to the conical shape illustrated in the figures. The upper portion defines a closed end of body 1. The base has an open lower end and at least the base and central portion are hollow. A battery housing 16 configured to properly align the corresponding terminals of battery 5 and motor 4 is preferably attached to motor 4 for displacement and removal from inside body 1. In exemplary embodiments, battery housing 16 is configured to encapsulate motor 4 for aesthetic appeal when viewing the same from outside body 1 which is clear in most cases. However, it is contemplated that body 1 may be fabricated with a colored glass so motor 4 is not visually discernable outside body 1 when disposed therein, thus negating a desire to encapsulate motor 4 with battery housing 16.

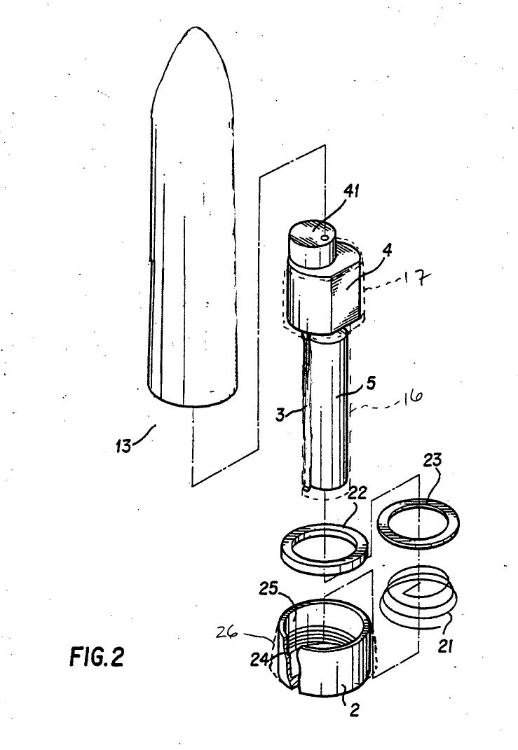

In such instances, the lower end of the motor 4 housing is secured to the upper end of the battery housing 16 by electronic fusion, adhesive or other suitable means, preferably waterproof. Motor 4 preferably includes a resilient or compliant sleeve 17 surrounding at least a leading portion of motor 4 (shown in phantom in FIG. 2). Sleeve 17 is configured to wedge against the walls defining the hollow of body 1 upon installation of motor 4 and housing 16 inside body 1. Sleeve 17 provides a resilient cushion like mounting which reduces noise.

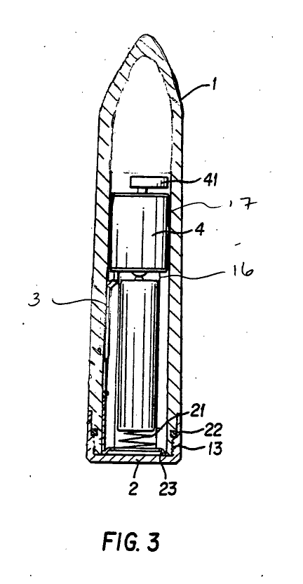

A trailing 13 may be arranged at a bottom of body 1 for fixing a cap or battery cover 2 by means of screw joint as indicated in FIG. 3. However, battery cover 2 is preferably configured to form a watertight seal with body 1 with a resilient press fit engagement therebetween. In a preferred embodiment, battery cover 2 is configured with a tapered cylindrical wall indicated with phantom lines 26 in FIG. 2 to wedge against an inside wall defining the hollow of body 1. However, as will be recognized by one skilled in the pertinent art, any battery cover 2 suitable for a watertight seal with body 1 is contemplated.

Referring to FIG. 2, the battery cover 2 has a conductive spring 21, a rubber washer 22, and a conductive ring plate 23. The rubber washer 22 is arranged at the top of a trapezoidal groove 24 inside the battery cover such that the rubber washer 22 is tightly in contact with body 1 when the battery cover 2 is removably secured with body 1 by being disposed over an exterior wall defining body 1. This arrangement provides a water proofing effect. An electrical conductor 3 extends inside the inner hollow chamber and outside along casing 16 to provide electrical connection between motor 4 and a battery 5. In an exemplary embodiment, electrical conductor is a contact strip or reed 3 having one end connected with the input terminal of the motor 4, and the other end operably connected with the anode of battery 5 via conductive ring plate 23. The trough 25 of battery cover 2 is configured for allocation of a spring 21. The lower part of spring 21 is connected to conductive ring plate 23. It will be recognized by one skilled in the art that the mechanical force provided by spring 21 is less than the force required to break the water proof seal between body 1 and cover 2. The top or upper part of the spring 21 is connected to the negative end of battery 5, such that the conductive ring plate 23 contacts the reed 3 extending to the open in body 1 to “turn on” the power at the time the battery cover 2 is assembled with the body. Rubber washer 22 provides a water proofing effect, however, resiliency of the walls defining cover 2 may also provide the same water proofing effect without having to use rubber washer 22. As the cover 2 is assembled with body 1, the electrical circuit is closed causing the motor shaft to rotate and which in turn causes the eccentric block 41 to revolve and vibrate.

When the motor 4 is in operation, it rotates the motor shaft extending therefrom and the eccentrically mounted weight 41. The eccentric mounting of the weight 41 imparts rapid vibration to the vibrator, such vibration preferably, but not necessarily, being the strongest proximate the conical portion of body 1. The vibrator and particularly these parts thereof may then be applied to any part of the body to convey the vibration to the body.

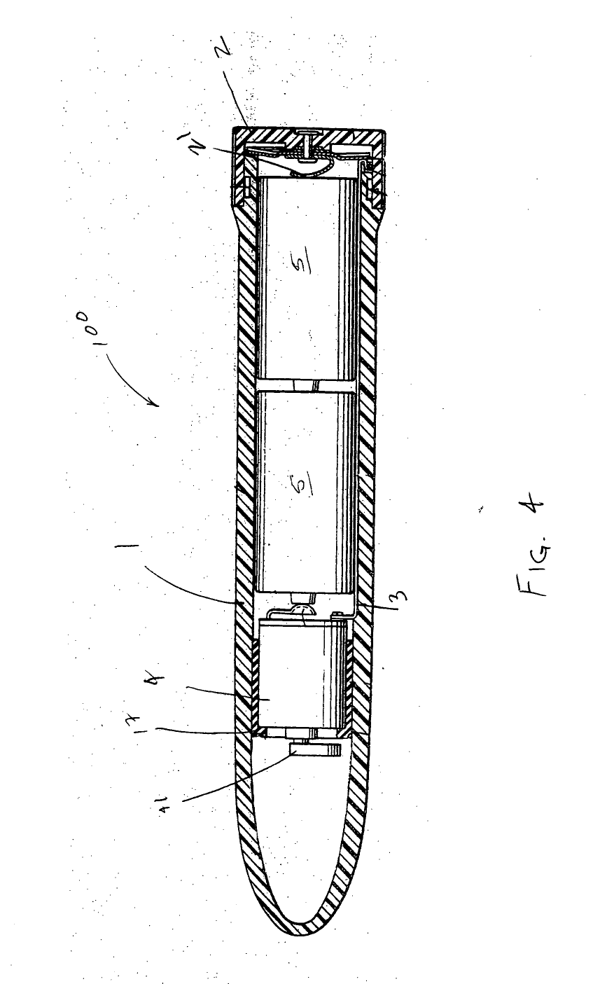

FIG. 4 illustrates an exemplary embodiment of a vibrator 100 having two batteries 5 in tandem. Battery housing 16 has been omitted for the sake of clarity. In a preferred embodiment, vibrator 100 employs two flashlight or AA batteries. Furthermore, it will be recognized that in further alternative embodiments of the invention which may incorporate the same contact strip 3 construction described above and in which the motor 4 can be selectively operated at either high or low speed, and therebetween, the speed selection is accomplished by manual rotation of a rotatable end cap of cover 2 as described in U.S. Pat. No. 3,549,920 to Cutler et al. and is incorporated by reference herewith in its entirety. Such speed selection control is well known in the art.

It will be noted that if the motor 4 or battery 5 should need to be removed for replacement or repair, the user need only remove battery cover 2 and turn the vibrator upside down while manually pulling on the battery housing 16 to break the wedged press-fit engagement between sleeve 17 and body 1. The motor 4 and housing 16 will then drop out as an assembly and a new motor 4 and or battery can be inserted in its place. Likewise, the same may be removed from body 1 in order to heat, cool, or clean by sterilization of body 1 as desired without causing damage to the motor and/or battery of the vibrator by being removed therefrom.

Referring again to FIG. 1, it will be recognized by one skilled in the pertinent art that a known polymeric vibrator may be disposed in body 1 with cap 2 removably sealing the polymeric vibrator in the hollow defined by body 1. In this manner, prior art polymeric vibrators can be safely used between different partners by removing the vibrator and sterilizing body 1 before use with another partner. Alternatively, body 1 may be simply cleaned with soap and water to remove any residual lubricant previously applied and provides an odorless body 1. The hollow of body 1 may be configured such that a conical portion of the polymeric vibrator wedges against a wall of body 1 defining the hollow. It is also contemplated that one or more polymeric sleeves or O-rings (e.g., sleeve 17) may be disposed over a thinner polymeric vibrator such that the polymeric vibrator operably contacts body 1 for vibration thereof. Further, it will be recognized that body 1 may be defined having a length suitable to provide watertight confinement therein of vibrators having different lengths. Lastly, it is also contemplated that corded polymeric vibrators may be disposed in body 1 without utilization of cap 2.

In all of the exemplary embodiments described above, body 1 of the sexual aid device is fabricated of a generally lubricous glass-based material. This glass-based material contains an appreciable amount of an oxide of boron to render it lubricious and resistant to heat, chemicals, electricity and bacterial absorption, while providing prolonged use and sufficient water-resistance for submersion in soap and water, and water-related uses. Body 1 is a substantially unitary one-piece waterproof glass shell. In preferred embodiments, body 1 includes a thickness of about 2 mm to about 5 mm, however, other thicknesses are contemplated that are suitable for its intended purpose. The combination of the vibrating mechanicals inside body 1 with cover 2 completing a water tight seal, the glass vibrator may be submerged under water for prolonged periods without water getting to the mechanical and electrical devices disposed inside.

The present invention is a stimulation device designed for anal and/or vaginal stimulation. Each of the embodiments of the present invention is hand-crafted as a solid glass-based tubular structure. As such, the device will stay slick for a long time without reapplying extra lubrication or rewetting. In addition, the devices will not hold odor. Each device is seamless, and created individually, one at a time. The devices are inspected carefully to insure perfect smoothness. If threads are chosen for securing the cap 2 therewith, it is contemplated that such threads may be hand tooled threads in body 1 using a graphite tool, for example. The devices may be sterilized, washed in soap and water, wiped with alcohol or autoclaved. The devices may be used with water and oil-based lubricants. Unlike conventional water-based lubricants used to protect latex condoms which become sticky when used on rubber-like devices, the present invention provides smooth contact with rectal tissues. This arrangement of components and materials renders the present invention very suitable for mass production.

As to the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

While preferred embodiments have been shown and described, various modifications and substitutions may be made thereto without departing from the spirit and scope of the invention, including the use of the geometries taught in other conventional vibrator assemblies. Further, since numerous modifications and changes will readily occur to those skilled in the art, it is not desired to limit the invention to the exact construction and operation shown and described, and accordingly, all suitable modifications and equivalents may be resorted to, falling within the scope of the invention. Accordingly, it is to be understood that the apparatus and intended use thereof have been described by way of illustration only, and such illustrations and embodiments as have been disclosed herein are not to be construed as limiting to the claims.

1. A vibrator comprising:
a housing having a generally cylindrical base and central portion and an upper portion defining a closed upper end, the base having an open lower end and at least said base and central portion being hollow, said housing being fabricated of a lubricious glass-based single piece of material;
an electric motor mounted in the hollow central portion of said housing;
vibrator means including a block and an eccentric drive therefore coupled to said motor;
battery means disposed in the hollow base of said housing, said battery means is operably coupled with said motor;
means for connecting said motor and said battery means in series circuit; and
an end sealing means including a cap mounted on the open end of said base for releasably sealing said open end so that the hollow interior of said housing is waterproof when said cap is mounted on said base, said end sealing means providing an external switch means operably coupled with said battery means and said motor for selectively energizing said motor to drive said block in eccentric rotation to thereby cause said housing to vibrate.

2. The vibrator of claim 1, wherein the cross-sectional diameter of the cap is such that the cap can be wedged intimately into the open lower end of the housing to provide a watertight seal of the hollow.

3. The vibrator of claim 1, wherein the housing is an elongated housing including a substantially tubular open channel defining the hollow.

4. The vibrator of claim 1, wherein a first sleeve surrounds at least a portion defining an exterior perimeter defining said motor, said first sleeve configured to reduce noise caused by vibration of said motor against said housing.

5. The vibrator of claim 4, wherein said first sleeve is a layer on the outside of said motor to hold the motor tight in the hollow.

6. The vibrator of claim 4, wherein said first sleeve is configured to be resilient to be caused to wedge said motor in said hollow.

7. The vibrator of claim 4, wherein a second sleeve is disposed around said battery means, said second sleeve configured to stabilize said battery means in said hollow by filling a space therebetween.

8. The vibrator of claim 1, wherein said battery means includes a battery housing operably coupled to said motor, said battery housing configured to receive at least one D.C. battery therein and align a positive terminal thereof with a motor terminal.

9. The vibrator of claim 7 wherein said first and second sleeves are formed of a material having a durometer hardness of about 50-100.

10. The vibrator of claim 1, wherein said battery means and said motor are configured for removal from said housing.

11. The vibrator of claim 1, further comprising:
switch means incorporated with said cap to close said circuit.

12. The vibrator of claim 11, wherein said switch means is configured to vary a speed of said motor.

13. The vibrator of claim 1, wherein said glass-based single piece of material includes an oxide of boron.

14. A watertight battery operated glass vibrator comprising a hollow sleeve head, a head extending and integrally formed with said sleeve, and communicating with the interior of said head, an electric motor in said sleeve provided with an axial drive shaft, and projecting into said head, battery means in said sleeve in tandem with and behind said motor, a cap sealably mounted on an open end of said sleeve of said sleeve forming a watertight seal in side said sleeve, means to connect said battery means in series circuit with said motor, and switch means on said cap configured to control said circuit, and an eccentric weighted means fixed on the motor shaft, said head and sleeve being fabricated of a lubricious glass-based single piece of material.

15. The vibrator of claim 14, wherein said glass-based single piece of material includes an oxide of boron.

16. The vibrator of claim 14, wherein said cap being removably mounted at the open end of said sleeve proximate said battery means and acting to hold a battery in said sleeve.

17. The vibrator of claim 14, further comprising an O-ring disposed between said cap and sleeve to render the sleeve watertight.

18. The vibrator of claim 14, wherein said head and sleeve are configured and dimensioned as a phallus.

19. A cordless electric vibrator for use on the human body, said vibrator comprising a housing, said housing being fabricated of a lubricious glass-based single piece of material, an electrically operated motor disposed within said housing, a shaft rotatably connected to said motor, an eccentrically mounted weight carried by said shaft to impart vibration to said housing upon the operation of said motor, at least one dry cell battery disposed within said housing, a contact strip connected at one end thereof to said motor, a cap mounted to create a watertight seal on said housing and acting as a switch to control the operation of said motor by engaging said contact strip to complete an electrical circuit through said battery and motor.

20. The vibrator of claim 19, wherein said housing being elongated and substantially cylindrical, said housing having a closed end and an open end, said motor being disposed proximate to said closed end, said end cap being removably mounted on said open end of said housing, said end cap being disposed adjacent to said battery and acting to hold said battery within said housing.

21. A sexual aid device comprising:
a housing having a generally cylindrical base and central portion and an upper portion defining a closed upper end, the base having an open lower end and at least said base and central portion being hollow, said housing being fabricated of a lubricious glass-based single piece of material;
a vibrator mounted in the hollow central portion of said housing; and
an end sealing means including a removable cap mounted on the open end of said base for releasably sealing said open end so that the hollow interior of said housing is waterproof when said cap is mounted on said base, said vibrator causing said housing to vibrate.

22. The sexual aid device of claim 21, wherein said glass-based single piece of material includes an oxide of boron.

23. The sexual aid device of claim 21, wherein the cross-sectional diameter of the cap is such that the cap can be wedged intimately into the open lower end of the housing to provide a watertight seal of the hollow.

24. The sexual aid device of claim 21, wherein the cross-sectional diameter of the cap is such that the cap can be fitted intimately over the open lower end of the housing to provide a watertight seal of the hollow.

25. The sexual aid device of claim 21, wherein said vibrator is one of:
a corded vibrator;
a polymeric vibrator;
a self-contained vibrator; and
a vibrator having at least one sleeve or O-ring around a perimeter defining the vibrator, said sleeve and O-ring configured to provide operable contact between the vibrator and the housing.
